(19) United States
(12) Patent Application Publication
(10) Pub. No.: US 2024/0350703 A1
(43) Pub. Date: Oct. 24, 2024

(54) TOUGH ANTIBACTERIAL HYDROGEL DRESSING AND PREPARATION METHOD THEREOF

(71) Applicants: ZHEJIANG UNIVERSITY, Hangzhou (CN); SHAOXING RESEARCH INSTITUTE OF ZHEJIANG UNIVERSITY, Shaoxing (CN)

(72) Inventors: Changyou GAO, Hangzhou (CN); Xiaoqing LIU, Hangzhou (CN); Qiuyi WANG, Hangzhou (CN)

(21) Appl. No.: 18/757,627

(22) Filed: Jun. 28, 2024

Related U.S. Application Data
(63) Continuation of application No. PCT/CN2022/097164, filed on Jun. 6, 2022.

(30) Foreign Application Priority Data
May 30, 2022 (CN) 202210600570.9

(51) Int. Cl. A61L 26/00

(52) U.S. Cl. CPC A61L 26/0066; A61L 26/008; A61L 2300/406

(57) ABSTRACT

A tough antibacterial hydrogel dressing and a preparation method thereof provided. The hydrogel dressing is gelatinized based on hydrogen bonding of gelatinizing skeleton such as polyvinyl alcohol, gelatin, collagen, etc. Before gelatinizing, hyperbranched polylysine is mixed, and after gelatinizing, the mechanical properties are enhanced by soaking in an ionic solution. Physical gelatinizing by hydrogen bonding can avoid the toxicity problem caused by residual crosslinking agent in the chemical crosslinking method, and the shortcoming of poor mechanical properties of hydrogel obtained by physical crosslinking method is overcome by Hoffmeister effect salting-out. The hydrogel dressing prepared has good tensile and compressive properties and antibacterial properties. The hydrogel dressing provided is suitable for various skin wounds, and has the advantages of simple preparation method, high repeatability and low cost, and is expected to be widely used in the field of antibacterial skin dressings.

### TECHNICAL FIELD

The present application relates to the technical field of biomedical polymer materials, in particular to a tough antibacterial hydrogel dressing and a preparation method thereof.

### BACKGROUND

Skin injury is a serious threat to human health, which will lead to the loss of water and heat in the body and reduce the ability to resist the invasion of external pathogens. Wound healing includes four processes: hemostasis, inflammation, proliferation and remodeling. The whole healing process will last for several days to several years, depending on the degree of injury, age and health status of patients, as well as external factors such as foreign bodies and infections. Skin injuries are frequent, and the healing of skin wounds is a common clinical problem. It costs a lot to treat wounds, especially chronic wounds, which brings great economic pressure to patients and medical system. In order to reduce the cost of wound care and solve the problems of exudation, infection and scar formation in the process of wound healing, we continue to develop functional wound dressings. With the gradual clarification of wound healing theory, the form of wound dressing has also undergone great changes. Traditional wound dressings, such as gauze, bandage, cotton pad and the like are dry dressings, which can only provide physical protection for the wound and have limited effect on wound healing and infection prevention, and the adhesion to the wound when the dressing is removed will cause secondary damage; modern dressings, such as foams, hydrocolloids and hydrogels, have the advantages of debridement, moisturizing, anti-infection and scar inhibition compared with traditional dressings based on the healing theory in humid environment. Among them, hydrogels have attracted wide attention because of their good permeability, moisturizing, non-adhesion, biocompatibility and being used as carriers.

However, hydrogel dressings also have their limitations. Hydrogel is a hydrophilic three-dimensional macromolecular network, which is obtained by cross-linking between polymer chains. The cross-linking methods can be divided into physical cross-linking and chemical cross-linking. Chemical crosslinking requires the introduction of cross-linking agents and organic solvents in some cases, but these cross-linking agents and organic solvents cannot be completely removed from the system, and the residues are easy to cause toxicity problems. Physical crosslinking methods mainly include hydrogen bonding, van der Waals force, subject-guest interaction, electrostatic interaction. Although physical crosslinking avoids the residual crosslinking agent and organic solvent, the obtained hydrogel usually has poor mechanical properties and cannot meet the needs of daily activities of skin wounds, or its preparation process is complicated and energy consumption is high, such as cyclic freezing and thawing. How to avoid the residue of cross-linking agent and organic solvent, and ensure that hydrogel has good mechanical properties, and the preparation method is simple and cheap, is a big problem in the field of hydrogel dressing.

On the other hand, the moisturizing property of hydrogel dressing can provide a moist and healing environment for wounds, but it also provides an excellent place for bacteria to breed. At present, most hydrogel dressings on the market are powerless against bacterial infection, and the general improvement method for this problem is to add antibacterial agents to endow hydrogel dressings with antibacterial ability. Antibacterial agents can be divided into natural antibacterial agents, inorganic antibacterial agents and organic antibacterial agents according to different sources, and the typical representatives are chitosan, silver ions and antibiotics respectively. Chitosan is widely available, green and safe, but its antibacterial ability is weak due to the small number of effective antibacterial groups. Silver ions have long effective action time and no drug resistance, but most of them are added to hydrogel in nano form, which has poor stability, easy reunion and poor killing effect on gram-positive bacteria. Antibiotics are a kind of common and effective antibacterial agents, but their single-target binding mechanism with bacterial surface proteins easily leads to drug resistance of bacteria. Therefore, researchers are eager to find a new antibacterial hydrogel dressing that is safe, efficient, broad-spectrum antibacterial, stable in properties, easy to obtain, low in cost, non-toxic and not tending to induce drug resistance. One way of thinking is to find an antibacterial agent rich in amino groups, and the antibacterial agent should have the ability to be easily mixed into the hydrogel and quickly released in application.

### SUMMARY

In view of the above problems, the object of the present application is to provide a tough antibacterial hydrogel dressing and a preparation method thereof. The hydrogel dressing enhances the mechanical properties through salting-out on the basis of physical crosslinking, and the preparation process is simple and easy to repeat; hyperbranched poly-lysine, a new and efficient antibacterial agent, has excellent antibacterial properties, which can be used for antibacterial and healing of skin wounds.

In order to achieve the above purpose, the present application provides the following technical solutions: a method for preparing a tough antibacterial hydrogel dressing, which specifically includes the following components:

5-20 parts of a main material, 20-50 parts of glycerol, and 0.0025-0.3 part of hyperbranched polylysine, with the balance being water, per 100 parts by weight of the dressing.

Provided is a method for preparing the tough antibacterial hydrogel dressing, and the preparing process includes the following steps:
adding the main material and hyperbranched polylysine into a mixed solution of water and glycerol, stirring and heating to full dissolution; standing the dissolved solution to be gelatinized; cutting solid hydrogel into a required shape, soaking in an ionic solution of 10-35 wt % for 8-48 hours, taking out, washing with water and wiping.

Further, the main material in the preparing process is a gelatinizing skeleton material which is gelatinized by hydrogen bonding.

Further, the main material in the preparing process is a combination of one of more of chitosan, collagen, alginate, hyaluronic acid, polyethylene glycol, gelatin, polyurethane, polylactic acid, polyvinylpyrrolidone, polyvinyl alcohol, polyacrylic acid and carbomer.

Further, the heating is conducted at a condition of 10-60 minutes at 40-80° C., and the standing is conducted at a condition of 30-60 minutes at room temperature or 5-15 minutes at 4° C.

Further, the ionic solution in the preparing process is composed of an anion, which is one of tartrate, acetate, chromate, citrate, sulfate, bisulphate, carbonate, bicarbonate, dihydrogen phosphate, thiosulfate and chloride, and a cation, which is one of ammonium ion, lithium ion, potassium ion, sodium ion, manganese ion, calcium ion and barium ion.

Compared with the prior art, the present application has the following beneficial effects:

1. The tough antibacterial hydrogel dressing provided by the present application is gelatinized by the hydrogen bond of the main material, thus avoiding the toxicity problem caused by the addition of the cross-linking agent, and still maintaining the block shape.

2. The tensile and compressive properties of the tough antibacterial hydrogel dressing provided by the present application are greatly improved through salting-out enhancement of Hoffmeister effect, which exceeds the strength level of the existing dressing by tens of kPa.

3. The tough antibacterial hydrogel dressing provided by the present application contains less antibacterial agent than the prior art; the hyperbranched polylysine can play an effective and broad-spectrum antibacterial and bactericidal role by destroying bacterial cell membrane and DNA and improving the mechanism of active oxygen and water equality in bacterial cells.

4. The preparation method of the tough antibacterial hydrogel dressing provided by the present application has the advantages of simple process, simple operation, low cost and good repeatability, and the obtained hydrogel dressing is suitable for various skin wounds such as mechanical injury wounds, thermal injury wounds, ulcerative wounds and the like.

### BRIEF DESCRIPTION OF DRAWINGS

FIG. 1 is an illustration of a hydrogel dressing prepared by the present application;

FIG. 2 shows the mechanical properties of the hydrogel dressings in Example 2 and Comparative Example 1, in which (a) and (b) are compression test curves of the hydrogel dressings in Comparative Example 1 and Example 2 respectively, and (c) and (d) are tensile test curves of the hydrogel dressings in Comparative Example 1 and Example 2 respectively;

FIG. 3 is an apparent view of the inhibition zone of the hydrogel dressing in Example 3;

FIG. 4 shows in vitro antibacterial properties of the hydrogel dressings in Example 4 and Comparative Example 2.

### DESCRIPTION OF EMBODIMENTS

The technical solution of the present application will be further explained with examples, but these examples are not intended to limit the present application.

### Example 1

Step 1: 25 mg of hyperbranched polylysine (3 kDa) and 5 g of collagen were weighed, and were then added into a mixed solution of 24.995 mL of ultrapure water and 20 mL of glycerol, and the mixture was stirred at 60° C. for 1 hour to be dissolved.

Step 2: the dissolved solution was allowed to stand at room temperature for 60 minutes to be gelatinized.

Step 3: the hydrogel was cut into pieces, which were immersed in 15% sodium dihydrogen phosphate solution for 24 hours, then taken out, rinsed with deionized water and wiped.

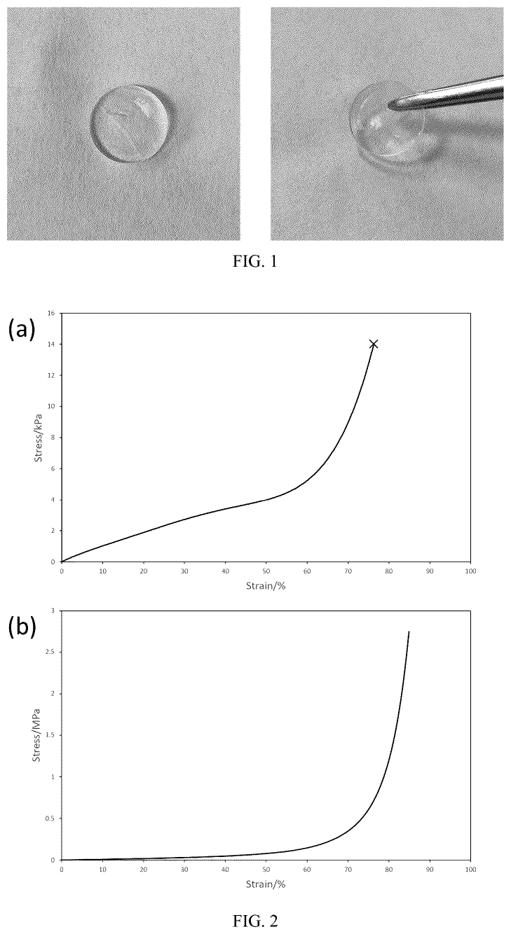

The display diagram of the hydrogel prepared in this example is shown in FIG. 1.

### Example 2

Step 1 50 mg of hyperbranched polylysine (3 kDa) and 5 g of gelatin were weighed, and are added into a mixed solution of 24.95 mL of ultrapure water and 20 mL of glycerol, and the mixture was stirred at 50° C. for 30 minutes to be dissolved.

Step 2: the dissolved solution was allowed to stand at 4° C. for 20 minutes to be gelatinized.

Step 3: the hydrogel was formulated into rectangular strips and columns, which were immersed in 20% ammonium sulfate solution for 12 hours, then taken out, rinsed with deionized water and wiped.

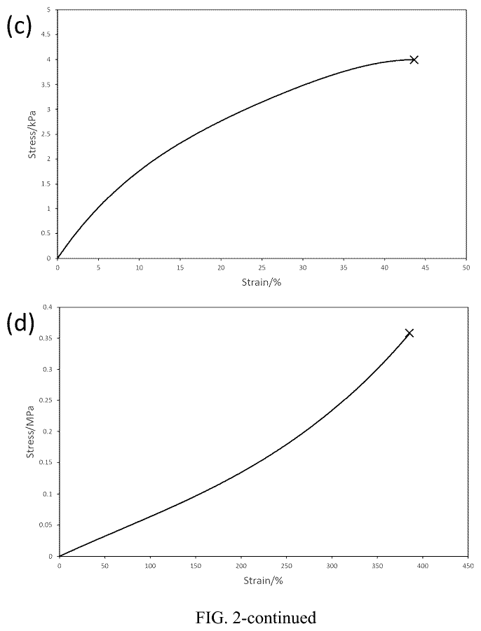

The following is the mechanical properties test of the hydrogel prepared in this example. At a compression speed of 2 mm/min, the compressive property of the hydrogel sample was tested, as shown in FIG. 2(b): the tensile property of the hydrogel sample was tested at a tensile speed of 10 mm/min, as shown in FIG. 2(d). It can be seen that the hydrogel dressing prepared by this example has excellent mechanical properties, and it will not be damaged when it is compressed to 85% strain, and the maximum stress reaches 2.8 MPa, and it can be recovered when the load deformation is removed; the tensile strength can reach 0.37 MPa, the elongation at break is 397%, and the elastic modulus is 0.17 MPa.

### Example 3

Step 1: 25 mg of hyperbranched polylysine (5 kDa) and 5 g of collagen were weighed, and added into a mixed solution of 34.95 mL of ultrapure water and 10 mL of glycerol, and the mixture was stirred at 60° C. for 1 h to be dissolved.

Step 2: the dissolved solution was allowed to stand at room temperature for 60 minutes to be gelatinized.

Step 3: the hydrogel was cut into pieces, which were immersed in 15% sodium citrate solution for 12 hours, then taken out, rinsed with deionized water and wiped.

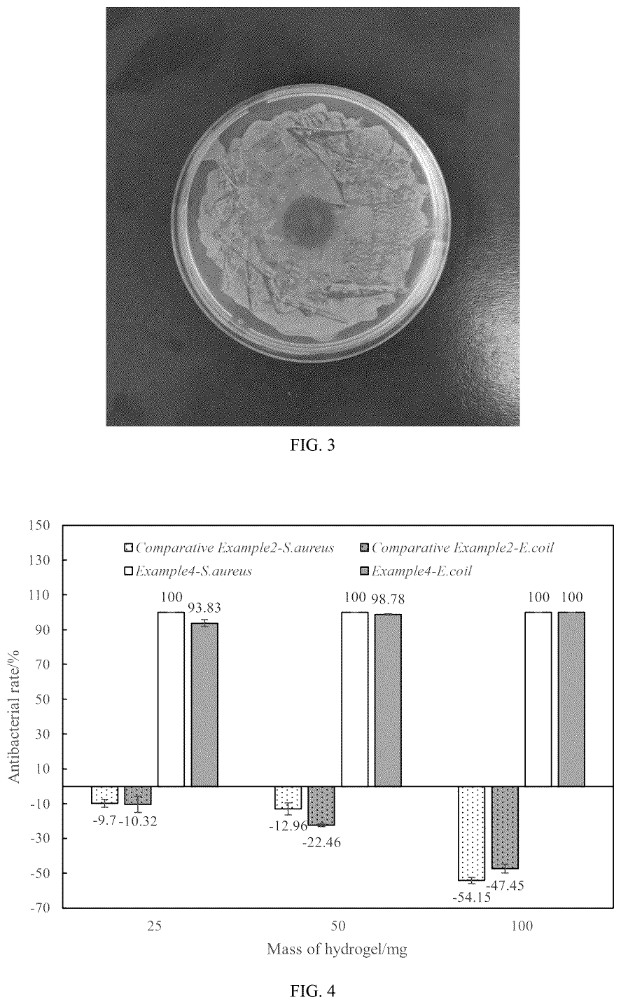

The following is the inhibition zone test of the hydrogel prepared in this example. A hydrogel disc with a diameter of 6 mm was placed on an agar medium coated with 200 μL of Staphylococcus aureus with a concentration of 10⁸ CFU/mL, and cultured at 37° C. for 12 hours. The apparent diagram of bacteriostatic circle obtained is shown in FIG. 3, and it can be seen that an obvious inhibition zone appeared around the hydrogel.

### Example 4

Step 1: 50 mg of hyperbranched polylysine (5 kDa) and 5 g of gelatin were weighed, added into a mixed solution of 34.995 mL of ultrapure water and 10 mL of glycerol, and then the mixture was stirred at 50° C. for 30 min to be dissolved.

Step 2: the dissolved solution was allowed to stand at 4° C. for 20 minutes to be gelatinized.

Step 3: the hydrogel was cut into pieces, which were immersed in 20% sodium citrate solution for 12 hours, then taken out, rinsed with deionized water and wiped.

The following is an in vitro antibacterial test of the hydrogel prepared in this example. Hydrogels with different qualities (25 mg, 50 mg, 100 mg) were co-cultured with 500 μL of Staphylococcus aureus and Escherichia coli with a concentration of 10⁶ CFU/mL at 37° C. for 12 hours, and the number of colonies was quantified on agar plates by a smear method. The obtained bacteriostatic rate is shown in FIG. 4. As can be seen from FIG. 4, due to the loaded hyperbranched polylysine, the hydrogel prepared in this example achieved 100% killing of Staphylococcus aureus at three dosage levels; for Escherichia coli, it has obvious killing effect at the dosages of 25 mg and 50 mg, and can achieve 100% killing at the dosage of 100 mg.

### Comparative Example 1

Step 1: 50 mg of hyperbranched polylysine (3 kDa) and 5 g of gelatin were weighed, and added into a mixed solution of 24.95 mL of ultrapure water and 20 mL of glycerol, and the mixture was stirred at 50° C. for 30 minutes to be dissolved.

Step 2: the dissolved solution was allowed to stand at 4° C. for 20 minutes to be gelatinized.

Step 3: the hydrogel was formulated into rectangular strips and columns.

The following is the mechanical property test of the hydrogel prepared in this comparative example. At a compression speed of 2 mm/min, the compressive property of the hydrogel sample was tested, as shown in FIG. 2(a). The tensile property of the hydrogel sample was tested at a tensile speed of 10 mm/min, as shown in FIG. 2(c). It is known that the hydrogel dressing has poor mechanical properties, and it will break when it is compressed to 76% strain, and the maximum stress is only 13.7 kPa; the tensile strength is only 4.0 kPa, the elongation at break is 44% and the elastic modulus is 0.03 MPa.

In the preparation process disclosed by the present application, the step of Hoffmeister effect salting-out enhancement is introduced, and the comparison of mechanical properties shown in FIG. 2 shows that the hydrogel dressing obtained by the preparation method disclosed by the present application has excellent mechanical properties.

### Comparative Example 2

Step 1: 5 g gelatin was weighed, and added into a mixed solution of 35 mL ultrapure water and 10 mL glycerol, and the mixture was stirred at 50° C. for 30 min to be dissolved.

Step 2: the dissolved solution was allowed to stand at 4° C. for 20 minutes to be gelatinized.

Step 3: the hydrogel was cut into pieces, which were immersed in 20% sodium citrate solution for 12 hours, then taken out, rinsed with deionized water and wiped.

The following is the in vitro antibacterial performance test of the hydrogel prepared in this comparative example. Hydrogels with different qualities (25 mg, 50 mg, 100 mg) were co-cultured with 500 μL of Staphylococcus aureus and Escherichia coli with a concentration of 10⁶ CFU/mL at 37° C. for 12 hours, and the number of colonies was quantified on agar plates by a smear method. The obtained bacteriostatic rate is shown in FIG. 4. As can be seen from FIG. 4, the hydrogel prepared in this comparative example has no inhibitory effect on Staphylococcus aureus and Escherichia coli at three dosages, and even the added gelatin can provide nutrition for bacteria, resulting in an increase in the number of colonies, and the more the hydrogel is added, the more the number of colonies increases.

From the comparison of antibacterial properties shown in FIG. 3 and FIG. 4, it can be seen that the hydrogel dressing containing hyperbranched polylysine disclosed by the present application has excellent antibacterial properties.

What is claimed is:

1. A tough antibacterial hydrogel dressing, comprising 5-20 parts of a main material, 20-50 parts of glycerol, and 0.0025-0.3 part of hyperbranched polylysine, with the balance being water, per 100 parts by weight of the dressing.

2. A method for preparing the tough antibacterial hydrogel dressing according to claim 1, comprising: adding the main material and hyperbranched polylysine into a mixed solution of water and glycerol, stirring and heating to full dissolution; standing the dissolved solution to be gelatinized; cutting solid hydrogel into a required shape, soaking in an ionic solution of 10-35 wt % for 8-48 hours, taking out, washing with water and wiping; wherein the main material is a gelatinizing skeleton material which is gelatinized by hydrogen bonding.

3. The method for preparing the tough antibacterial hydrogel dressing of claim 2, wherein the main material is at least one of chitosan, collagen, alginate, hyaluronic acid, polyethylene glycol, gelatin, polyurethane, polylactic acid, polyvinylpyrrolidone, polyvinyl alcohol, polyacrylic acid and carbomer.

4. The method for preparing the tough antibacterial hydrogel dressing of claim 2, wherein the hyperbranched polylysine has a molecular weight of 3-7 kDa.

5. The method for preparing the tough antibacterial hydrogel dressing of claim 2, wherein the heating is conducted at a condition of 10-60 minutes at 40-80° C., and the standing is conducted at a condition of 30-60 minutes at room temperature or 5-15 minutes at 4° C.

6. The method for preparing the tough antibacterial hydrogel dressing of claim 2, wherein the ionic solution is composed of an anion which is one of tartrate, acetate, chromate, citrate, sulfate, bisulphate, carbonate, bicarbonate, dihydrogen phosphate, thiosulfate and chloride, and a cation which is one of ammonium ion, lithium ion, potassium ion, sodium ion, manganese ion, calcium ion and barium ion.

7. The tough antibacterial hydrogel dressing of claim 1, wherein the hyperbranched polylysine is loaded in the hydrogel dressing by physical blending.

8. The tough antibacterial hydrogel dressing of claim 1, wherein the dressing is able to apply to various wounds such as mechanical injury wound, thermal injury wound and ulcerative wound.
